(19) United States
(12) Patent Application Publication
(10) Pub. No.: US 2012/0202864 A1
(43) Pub. Date: Aug. 9, 2012

(54) Compositions and Methods for Treatment of Glaucoma

(75) Inventor: Gerald Horn, Deerfield, IL (US)

(73) Assignee: ALPHA SYNERGY DEVELOPMENT INC., Dana Point, CA (US)

(21) Appl. No.: 13/365,138

(22) Filed: Feb. 2, 2012

Related U.S. Application Data
(63) Continuation-in-part of application No. 12/931,632, filed on Feb. 3, 2011.

(51) Int. Cl. A61K 31/4174; A61P 27/06

(52) U.S. Cl. 514/396

(57) ABSTRACT

The invention provides α-2 adrenergic receptor agonist compositions and methods for treating glaucoma and other intraocular conditions. The preferred α-2 agonist used in the inventive compositions and methods is dexmedetomidine at acidic pH and extremely low concentrations.

This application is a continuation-in-part of U.S. patent application Ser. No. 12/931,632, filed on Feb. 3, 2011. The entire teachings of the above-referenced application are incorporated herein by reference.

### BACKGROUND OF THE INVENTION

Glaucoma is a multifactorial disease which encompasses a spectrum ranging from elevated intraocular pressure (IOP) to reduced vascular perfusion of the optic nerve.

While many factors have been implicated as contributing causes of glaucoma, currently existing treatments for glaucoma have limited effectiveness in lowering IOP and/or are accompanied by a number of side effects, such as fatigue, sedation, lid allergy, topical allergy, and/or redness.

Because of the side effects, an additional major problem in glaucoma therapy is patient compliance in taking medications as prescribed. It is believed that many of these side effects and suboptimal efficacy of the existing treatments are unintended consequences of alpha-1 (α-1) receptor induction from treatment with alpha agonists.

It has been demonstrated that a 10% reduction in the risk of visual field loss progression is associated with each 1 mm Hg of intraocular pressure reduction (Early Manifest Glaucoma Trial, Leske et al, 2003). Yet despite the development of lower concentration equally effective brimonidine formulations (for example, Alphagan® P 0.1% vs. brimonidine 0.2%), the percentage of patients with one or more treatment-related adverse events was still high—41.4% vs. 53%. (Cantor, Brimonidine in the treatment of glaucoma and ocular hypertension, Ther Clin Risk Manag. 2006 December; 2(4):337-346). Poor compliance can also lead to treatment failure, as up to 80% of glaucoma patients may not take their medication as prescribed. (Olthoff et al, Noncompliance with ocular hypotensive treatment in patients with glaucoma or ocular hypertension an evidence-based review, Ophthalmology. 2005 June; 112(6):953-61).

Prior art α-2 agonist glaucoma therapy with the most recent commercially available α-2 agonist brimonidine demonstrates significant loss of compliance. In one long term study, 36.4% of brimonidine treated patients dropped out within one year, while the corresponding figure for the beta blocker group (timolol) group was 10.1%. Further, in treatment of low tension glaucoma at a baseline mean IOP of 15, only a mean lap reduction to 14-a 6.6% mean IOP reduction—was obtained over a four year period. (Krupin, J. M. et al, A randomized trial of brimonidine versus timolol in preserving visual function: Results from the Low-pressure Glaucoma Treatment Study, American Journal of Ophthalmology 2011; 151: 671-681). Other studies have shown a maximum 18.7% IOP reduction for a mean IOP of 17. Gandolfi S A, et al, Effect of brimonidine on intraocular pressure in normal tension glaucoma: a short term clinical trial, Eur J. Ophthalmol. 2003 August-September; 13(7):611-5.

Prior art attempts to use dexmedetomidine were studied in normotensive and laser induced trabeculoplasty acute postoperative IOP spike suppression in a rabbit model. Only modest IOP reduction in normotensive (<21 mm Hg) eyes was obtained. Dexmedetomidine 0.005% and 0.05%, formulated with phosphate buffer to pH 6.4 was instilled.

Accordingly, there is a need for novel formulations of alpha-2 (α-2) agonists for the treatment of glaucoma which would have minimal, if any, cross-activation of α-1 receptors, may have more effective lap lowering, and with significantly reduced or eliminated side effects of conventional α-2 agonists, such as sedation and redness. In addition, an improved cosmetic appearance via both reduced redness and a cosmetically pleasing whiter shading of the eye may also reduce noncompliance.

### SUMMARY OF THE PRESENT INVENTION

The present invention provides compositions and methods effective for the treatment of glaucoma in a patient in need thereof. Preferably, the compositions of the invention are formulated to prevent sedation, eliminate or reduce redness, eliminate or reduce ocular allergy, as well as significantly reduce intraocular pressure.

In some embodiments, the provided compositions may also have an eye whitening effect. Most preferably, the compositions include all of the above benefits and also have neuroprotective benefits and may be used for optic nerve protection, including the treatment of neurodegenerative conditions, such as ischemic optic neuropathy, diabetic retinopathy, optic ischemia, retinal vascular ischemia, and other optic neuropathies, particularly those involving retinal ganglion cells and/or axons at or near the optic nerve lamina.

The present invention optimizes α-2 agonist corneal permeation utilizing a highly selective α-2 agonist which is formulated to have a high intraocular lipophilicity of preferably 2.5 or greater and range of topical lipophilicity of preferably 1.0 to 2.2.

The preferred compositions of the invention employ selective α-2 adrenergic receptor agonists which share some or all of the following characteristics:
a) a high selectivity for α-2 over α-1 adrenergic receptors, such as 1000:1 or greater; more preferably 1500:1 or greater; and even more preferably 2000:1 or greater;
b) a very low concentration, such as from between about 0.0075% to about 0.075%; more preferably, between about 0.020% to about 0.040% weight by volume;
d) a relatively acidic pH on topical delivery of between about 4.0 and 6.2, preferably between 4.5 and 6.0, and more preferably of between about 4.8 and 5.5; and
e) a high degree of intraocular lipophilicity as measured by the Log P, the equilibrated intraocular pH at 7.4, with an octanol-water partition coefficient Log P of between about 2.50 and 4.0; and more preferably between about 2.90 and 3.50 at physiologic pH.

Preferably, the compositions of the invention contain corneal penetration enhancers. Corneal penetration agents include, but are not limited to, citrate, a citrate salt and/or other salts which increase solubility, chelating agents, preservatives, ion-channeling agents, cyclodextrin, or other additives which increase corneal permeability.

It is currently believed that the most preferred selective α-2 adrenergic receptor agonist suitable for purposes of the invention is dexmedetomidine in specific formulations which meet the above-listed characteristics. Accordingly, in some embodiments, compositions and methods of the invention include dexmedetomidine, or another selective α-2 adrenergic receptor agonist, at a concentration from between about 0.0075% to about 0.075% weight by volume; more preferably, between about 0.015% to about 0.040% weight by volume; and even more preferably between about 0.025% and about 0.035% weight by volume.

For ophthalmic drug delivery, the ideal Log P value (octanol-water partition coefficient at pH 7.4, where the “minus” sign signifies hydrophilicity and the “plus” sign signifies lipophilicity) is between +2.0 and +3.0. The Log P value is highly drug/drug subclass specific, and while predictive software algorithms have been developed, there is no completely accurate means for determining the ideal Log P value for a proposed drug formulation. Further, to the best of the inventor's knowledge, determining the topical pH of a formulation for the optimal Log D value has only been attempted for brimonidine, where alkaline pH was preferred. The Log P value, however, is the octanol-water coefficient at pH 7.4, i.e., physiologic pH. The range between +2.0 and +3.0 typically allows for the best compromise between: a) the need for a highly lipophilic drug to penetrate the lipophilic corneal epithelium, and to a lesser extent, the very thin inner corneal membrane called Descemet's membrane, and b) a highly hydrophilic drug to penetrate the stroma, which is the middle layer of the corneal “sandwhich” that must be penetrated for effective ophthalmic absorption.

However, for any drug suitable for the purposes of the present invention, it has been discovered that the optimal pH of the formulation (i.e., the pH of the formulation before physiologic equilibration to pH 7.4) is such pH that results in a Log “D” value for the drug (the initial topical lipophilicity) of between 0.50 and 2.30, more preferably between 0.75 and 1.75, and still more preferably between about 1.0 and 1.50. The pH range of the formulation for these preferred Log D values is about 4.0 to 6.2, more preferably 4.5 to 5.7, and still more preferably 4.75-5.3. These formulation discoveries alone increase the percent IOP reduction from a formulation pH of 7.4-7.8 (preferred pH for brimonidine as Alphagan“P”) by nearly 100% at a pH of 5.0. In some embodiments, dexmedetomidine, or another selective α-2 adrenergic receptor agonist, has an octanol-water partition coefficient Log P of between about 2.40 and 4.00; and more preferably, between about 2.50 and 2.90.

In the most preferred embodiment, the invention provides a pharmaceutical composition for the treatment of glaucoma which includes:
a. dexmedetomidine at a concentration from between about 0.020% to about 0.035% weight by volume; and
b. at a pH of about 4.75 to 5.5, with or without buffers.

The compositions of the invention may optionally include:
c. a corneal penetration/solubility enhancer, such as a salt selected from the group consisting of citrate, mesylate, hydrobromide/bromide, acetate, fumarate, sulfate/bisulfate, succinate, phosphate, maleate, nitrate, tartrate, benzoate, carbonate, and pamoate; preferably at a concentration of between about 0.1% and 0.5%, and more preferably between about 0.15% to 0.20%; and/or
d. carboxymethyl cellulose (CMC) at a concentration of between about 0.05% and about 0.5% weight by volume, most preferably at 0.1%; and/or
e. mannitol at a concentration of between about 1% and about 10% weight by volume, most preferably at 4% and/or
f. 2-hydroxypropyl-beta cyclodextrin at a concentration of between about 0.5% and about 5% weight by volume; and/or
g. Tween® 80 detergent (or other Tween® detergent), including polyethylene glycol, propylene glycol, polyvinyl alcohol and glycerin; and/or
h. preservatives, including solubility enhancers, such as methylparaben, propylparaben, benzalkonium chloride (BAK) and ethylenediaminetetraacetic acid (EDTA), preferably at a concentration of between 0.01% and 0.05%, most preferably 0.02%;
i. buffers to bring the pH to about 4.0 to 6.2, and more preferably to 5.5, including but not limited to acidic or near acidic buffers, such as acetate, citrate, phosphate, maleate and caprylate; and
j. mucoadhesives, including but not limited to xanthum gums, chitosan and its derivatives; eudragits (e.g. NE30D); pyrrolidines (PVP; methyl cellulose (MC), sodium carboxy methylcellulose (SCMD, hydroxypropyl cellulose (HPC) and other cellulose derivates; carbomers; and poloxamers, including but not limited to Poloxamer 407 (or its trade name Pluronic®F127) at a concentration range of 0.5% to 20%, more preferably 2% to 8%, and still more preferably 3% to 5%.
k. Addition of mucoadhesive stabilizers for Poloxamer gels, such that the formulation remains a stable liquid at room temperature (about 18-24° C.), only gelling at body temperature (about 30° C.), and may include polyethyelene glycols (PEGs), including but not limited to PEG 4000 and PEG 6000 (the former to lower gelling temperature and the latter to increase it, dependent on other formulation variables such as electrolyte and other solute concentrations); preferably in a range of 0.50% to 5%, and/or propylene glycol (PG) for its humectant properties (moisture retention).

The composition may further include other stabilizing agents and/or other additives as more fully described below.

In some embodiments, compositions and methods of the invention include selective α-2 adrenergic receptor agonists which have binding affinities (IC) for α-2 over α-1 receptors of 1000 fold or greater and are highly lipophilic, having an octanol-water partition coefficient of about 2.00 or greater.

In yet other embodiments, compositions and methods of the invention include selective α-2 adrenergic receptor agonists which have Kᵢ for α-2 over α-1 receptors of 1000 fold or greater and are at a concentration from between about 0.001% to about 0.035% weight by volume.

In some embodiments, compositions and methods of the invention include selective α-2 adrenergic receptor agonists which have Kᵢ for α-2 over α-1 receptors of 1500 fold or greater, are present at a concentration from between about 0.010% to about 0.040% weight by volume, and have pH of about 6.2 or less.

In some embodiments, the compositions of the invention may also include other therapeutic agents; however, the compositions are intended to be effective without the need for any other therapeutic agents, specifically including, but not limited to, α-1 antagonists.

The invention also provides methods of treating and/or preventing glaucoma with the provided compositions. The provided methods lower IOP in glaucoma patients, reduce redness, and provide eye whitening. The provided methods may also treat ischemic optic neuropathy and other neuropathies of various etiologies due to neuroprotective effects of the provided compositions.

### DETAILED DESCRIPTION OF THE INVENTION

### Definitions

The term “α-1 adrenergic receptor” refers to a G-protein-coupled receptor (GPCR) associated with the G_{q} heterotrimeric G-protein.

The term “α-2 adrenergic receptor” refers to a GPCR associated with the Gᵢ heterotrimeric G-protein.

The term “selective α-2 adrenergic receptor agonists” encompasses all α-2 adrenergic receptor agonists which have a binding affinity of 1000 fold or greater for α-2 over α-1 adrenergic receptors, and more preferably 1500 fold or greater. The term also encompasses pharmaceutically acceptable salts, esters, prodrugs, and other derivatives of selective α-2 adrenergic receptor agonists.

The term “dexmedetomidine” encompasses, without limitation, dexmedetomidine salts, esters, prodrugs and other derivatives.

The term “prodrug” refers to a compound that may be converted under physiological conditions to a biologically active compound.

As used herein, the term “composition” is intended to encompass a product comprising the specified ingredients in the specified amounts, as well as any product which results, directly or indirectly, from a combination of the specified ingredients in the specified amounts.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

The term “significant side effects” refers to substantial side effects of the treatment which include at least: a) sedation of a patient such that the patient feels sedated and becomes impaired or b) visually noticeable increase in redness of a patient's eye due to hyperemia.

The term “medicamentosa” refers to the inflammatory sequelae of α-1 agonist topical medications, particularly following topical ocular or nasal delivery, such as the development of increased vasodilation and hyperemia, in its less severe form referred to as “rebound”. requiring more frequent instillation of topical vasoconstrictor, resulting in a cyclically increasing ischemia and, eventually, a persistent adverse toxicity lasting weeks to months even after drug discontinuation.

### Embodiments of the Invention

The present invention provides formulations of highly selective α-2 agonists with high lipophilicity at physiologic pH (Log P of 2.0 to about 4.0), such as dexmedetomidine (Log P 2.89), which are able to penetrate into the eye at an optimized acidified pH. Prior art use of α-2 agonists to treat glaucoma, such as clonidinie, apraclonidine, and the only currently commercially available α-2 agonist, brimonidine, all have either low selectivity or low lipophilicity,

It is believed that the acidified pH for dexmedetomidine and other selective α-2 agonists suitable for the invention optimizes corneal absorption, so that the high lipophilicity at intraocular pH 7.4 creates maximal peak and duration of IOP reduction possibly related to the many pigmented intraocular structures, with increased depot absorption and diffusion, while the acidified pH of the formulations improves penetration of the lipophilic—hydrophilic—lipophilic corneal “sandwhich” of corneal epithelium, stroma, and endothelium. The corneal absorption is believed to be optimal at a Log D value of from about 0.50 to about 2.3 for α-2 agonists, where brimonidine is limited to about 0.50, achieved only at its most alkaline pH. Dexmedetomine can be pH-adjusted to achieve 0.75 to 2.90 Log D (formulation pH determined octanol-water coefficient), as it is highly lipophilic drug relative to brimonidine, in addition to being 1.5× more α-2 selective. When the drug is dexmedetomidine, the optimal Log D value is from 0.75 to 2.2, and more preferably is about 1.00 to 1.50 at a pH of about 4.7 to 5.3.

The provided compositions and methods are effective for the treatment of glaucoma. Preferably, the compositions of the invention are formulated to prevent sedation, eliminate or reduce redness, as well as more significantly reduce intraocular pressure than prior art formulations of α-2 agonists. Because a common side effect of glaucoma drugs and, particularly, brimonidine, is eye redness (20-25% rebound redness with long term use of brimonidine), reduction of redness confers an added important advantage found with the present invention. The compositions of the invention provide considerable IOP lowering improvement over prior art α-2 agonists, particularly for the eyes of glaucoma patients, may increase duration of therapeutic action and reduce the incidence of rebound hyperemia and/or other allergic reaction. They also further improve cosmetic appearance (for example, increasing whiteness and providing additional whitening) of the treated eyes, resulting in improved patients' compliance; and provide optic nerve protection, retinal ganglion cell neuroprotection, an increase in α-2 agonist concentration in the inner retinal plexiform, and additional neuroprotective benefits. They may also increase the outflow at the trabecular meshwork which is populated with endothelial cells and believed to be populated with α-2a receptor in humans.

In some embodiments, the compositions and methods of the invention significantly lower intraocular pressure while at the same time reducing redness and providing eye whitening. Some of the observed intraocular pressure lowering effects include:
1) onset within one hour;
2) peak effects of over 30%, and as great as 55.9% (see, Example 1, Formulation 7, Table 3) reduction over normotensive baseline mean IOP of 17.5 at 3 hours post instillation;
3) peak effects at about 3-3.5 hours, compared to 2 hours for brimonidine;
4) prolonged action with about a 19% reduction over baseline at 8 hours from a single dose; and even greater effect anticipated for α-2 agonist class with increased outflow in addition to aqueous production suppression with long term use of two weeks or greater; and still greater effect with preferred mucoadhesive additives and related inactive stabilizing agents to about 36.7% at 6 hours (see, Example 1, Formulation 7, Table 3);
5) improved cosmetic appearance via reduction of redness and in some cases cosmetic whitening that are further increased with mucoadhesive additives.

Further advantages of the inventive compositions and methods include a greatly reduced concentration range of about 0.007%:0.075% vs. conventional formulations of 1-2% for apraclonidine, 0.3% for clonidine, and 0.1-0.2% for brimonidine, with reduction of topical and systemic side effects associated with previous alpha 2 agonists (such as apraclonidine and brimonidine), including but not limited to oral dryness, ocular hyperemia, burning and stinging, headache, blurring, foreign body sensation, fatigue/drowsiness, conjunctival follicles, ocular allergic reactions, ocular pruritus, corneal staining/erosion, photophobia, eyelid erythema, ocular ache/pain, ocular dryness, tearing, upper respiratory symptoms, eyelid edema, conjunctival edema, dizziness, blepharitis, ocular irritation, gastrointestinal symptoms, asthenia, abnormal vision, muscular pain, lid crusting, conjunctival hemorrhage, abnormal taste, insomnia, conjunctival discharge, depression, hypertension, anxiety, palpitations/arrhythmias, nasal dryness and syncope. It is believed that a preferred embodiment containing Poloxamer 407 at 1%-10%, and more preferably, 1-3%, further minimizes systemic side effects as demonstrated in the examples below by reducing flow of the formulation with reduced nasolacrimal duct drainage and nasal and systemic absorption.

For the purposes of this application, the terms Poloxamer 407 and Pluronic®F127 are used interchangeably.

It is believed that the inventive formulations provide a combination of a very high α-2 selectivity and very low concentration to enhance glaucoma therapy by reducing and/or eliminating unintended stimulation of intraocular α-1 receptors; providing a much greater binding strength to α-2 receptors (higher α-2/α-1 ratio than apraclonidine, clonidine, or brimonidine); and increasing intraocular available drug via absorption into intraocular pigmented structures from which it can then be released via diffusion. Even low levels of α-1 receptor induction topically with intraocular diffusion, and/or directly at intraocular α-1 receptors (triggered in inverse proportion to the α-2 selectivity x the concentration) may induce sufficient generalized constriction of vasomotor tone, or otherwise induce ischemia or pro-inflammatory cytokines to substantially degrade efficacy of α-2 agonists to reduce their optimal efficacy for the treatment of glaucoma, particularly as intended use is almost always long term or chronic.

In order for a subclass of α-2 agonists of the present invention to have superior and previously unknown ocular hypotensive (i.e., reducing IOP) and other therapeutic benefits for the treatment of glaucoma (including all forms of open angle glaucoma, ocular hypertension, pseudoexfoliative glaucoma, and neovascular glaucoma), a specific optimized combination of high α2/α1 selectivity, a high degree of corneal penetration achieved via formulation modifications and optimized topical lipophilicity, high intraocular lipophilicity, a formulated optimized acidified pH range and an extreme low dose relative to brimonidine is preferred. While a drug that satisfies any of these characteristics may work to some degree, there is an enhanced amplified benefit achieved when most or all of these characteristics are met.

Accordingly, the present invention provides both enhanced intraocular α-2 receptor agonist effects and reduction or elimination of unintended adverse induction of α-1 receptors. It is believed that the reduction or elimination of significant side effects is possible because the inventive compositions do not activate α-1 receptors, increase intraocular penetration, and increase intraocular binding affinity to cell membranes while decreasing the topical concentration required via previous alpha 2 agonist drugs. Because the present invention maximizes the potential of α-2 agonists, it provides compositions and methods to treat glaucoma which do not require a second therapeutic agent, such as prostaglandins, prostanoids, carbonic anhydrase inhibitors, or even α-1 antagonists, although any or all second therapeutic agents may be added providing further efficacy.

Achieving intraocular α-2 effects without inducing α-1 topical or intraocular ischemic effect (i.e., restriction of blood supply) promotes the full spectrum of α-2 agonist activity benefits for treating glaucoma: 1) reduced level of pro-inflammatory cytokines; 2) reduced direct general α-1 induced ischemia to retinal ganglion cells and optic nerve fibers, to which the optic nerve, particularly along the lamina cribosa, may be extremely sensitive; 3) longer duration and much more profound aqueous synthesis reduction without α-1 induced vasoconstriction and attendant ischemia; and 4) greater cell membrane permeation to reach α-2 receptors in the ciliary processes and/or trabecular meshwork.

A preferred acidified range of pH allows for improved topical delivery of the highly lipophilic subclass of the suitable α-2 agonists. Once topical delivery proceeds through the cornea (over minutes or tens of minutes) into the anterior chamber, the drug will have equilibrated to physiologic pH of 7.4 and each drug's specific Log P value (for example, for dexmedetomidine Log P is about 3.0, while for brimonidine, Log P is about 0.79 to 1.75, see Advanced Chemistry Development Report, Drug Bank). The difference in the Log P values of dexmedetomidine and brimonidine represents about 100 to 300 times higher lipophilicity of dexmedetomidine versus brimonidine.

The preferred selective α-2 adrenergic receptor agonists share the following characteristics:
a) a high selectivity for α-2 over α-1 adrenergic receptors, of at least 1000:1 or greater; more preferably 1500:1 or greater; and even more preferably 2000:1 or greater;
b) a very low concentration, such as from between about 0.007% to about 0.070%; more preferably, from between about 0.020% to about 0.035% weight by volume; more preferably between about 0.025% weight by volume, and still more preferably about 0.035% weight by volume; and wherein more viscous or mucoadhesive formulations may safely and effectively be used at still higher concentrations of up to about 0.035% to 0.075%, and still more preferably 0.035% to 0.040% due to a reduced nasal absorption driven side effect profile;
d) acidity, such as an acidic or near acidic pH of between about 4.0 to 6.2; and more preferably between about 4.5 and about 5.3;
e) a high degree of intraocular lipophilicity as measured by the Log P at pH 7.4, (the octanol-water partition coefficient of between about 2.0 and 4.00; and more preferably, between about 2.5 and 3.5 at physiologic pH;
f) a formulated lipophilicity value—the topical Log D value—achieved via pH modification to within a range of about 0.75 to 2.20, and more preferred of about 1.0 to about 2.0 and still more preferred of about 1.25 to about 1.75, where such pH modification is within a well tolerated range from 4.0 to 6.2;
g) a substantially greater solubility in an aqueous solution than the required concentration range of 0.002% (0.02 mg/cc) to 0.02% (0.2 mg/cc) to allow for improved solubility and stability over a range of temperatures, where such solubility is preferably about 1 mg/cc or greater at the preferred acidic pH range, and where drug solubility is exponentially greater at low pH than for alkaline formulation.

It is currently believed that the most preferred selective α-2 adrenergic receptor agonist suitable for purposes of the invention is dexmedetomidine as either the HCl salt, or as the citrate salt. Other salts may similarly be substituted for the HCl.

The compositions of the invention may also optionally include:
a. a corneal penetration/solubility enhancer, such as a salt selected from the group consisting of citrate, mesylate, hydrobromide/bromide, acetate, fumarate, sulfate/bisulfate, succinate, phosphate, maleate, nitrate, tartrate, benzoate, carbonate, and pamoate; preferably at a concentration of between about 0.1% and 0.5%, and more preferably between about 0.15% to 0.20%; and/or
b. carboxymethyl cellulose (CMC) at a concentration of between about 0.05% and about 0.5% weight by volume, most preferably at 0.1%; and/or
c. mannitol at a concentration of between about 1% and about 10% weight by volume, most preferably at 4%; and/or
d. 2-hydroxypropyl-beta cyclodextrin at a concentration of between about 0.5% and about 5% weight by volume; and/or
e. Tween® 80 detergent (or other Tween® detergent), including polyethylene glycol, propylene glycol, polyvinyl alcohol and glycerin; and/or
f. preservatives, including solubility enhancers, such as methylparaben, propylparaben, benzalkonium chloride (BAK) and ethylenediaminetetraacetic acid (EDTA), preferably at a concentration of between 0.01% and 0.05%, most preferably 0.02%;
g. buffers to bring the pH to about 4.0 to 6.2, and more preferably to 5.5, including but not limited to acidic or near acidic buffers, such as acetate, citrate, phosphate, maleate and caprylate; and
h. mucoadhesives, including but not limited to xanthum gums, chitosan and its derivatives; eudragits (e.g. NE30D); pyrrolidines (PVP; methyl cellulose (MC), sodium carboxy methylcellulose (SCMD), hydroxypropyl cellulose (HPC) and other cellulose derivates; xantham gums, carbomers; and poloxamers, including but not limited to Poloxamer 407 at a concentration range of 0.5% to 20%, more preferably 2% to 8%, and still more preferably 3% to 5%; and
i. Addition of mucoadhesive stabilizers for Poloxamer gels, such that the formulation remains a stable liquid at room temperature (about 18-24° C.), only gelling at body temperature (about 30° C.), and may include PEGs, including but not limited to PEG 4000 and PEG 6000 (the former to lower gelling temperature and the latter to increase it, dependent on other formulation variables such as electrolyte and other solute concentrations); preferably in a range of 0.50% to 5%, and or PG for its humectant properties (moisture retention).

Administration of α-2 agonists at a too high concentration, (i.e., substantially close to 0.10%) can lead to α-1 induced adverse effects, including pressure α-1 induced spikes, ischemia, adverse cytokine increase, adverse neuronal degenerative effects, sedation and other undesired side effects, such as redness, hyperemia, systemic hypotension, bradycardia, etc. For drugs such as clonidine and apraclonidine, these effects are further exacerbated by reduced α-2/α-1 selectivity.

### Selectivity for α-2 Versus α-1 Adrenergic Receptors

The selective α-2 adrenergic receptor agonists have binding affinities (Kᵢ) for α-2 over α-1 receptors of 1000:1 or greater; more preferably 1500:1 or greater; and even more preferably 2000:1 or greater. It is well within a skill in the art to design an assay to determine α-2/α-1 functional selectivity. For example, potency, activity or EC₅₀ at an α-2A receptor can be determined by assaying for inhibition of adenylate cyclase activity. Furthermore, inhibition of adenylate cyclase activity can be assayed, without limitation, in PC12 cells stably expressing an α-2A receptor such as a human α-2A receptor. Additionally, potency, activity or EC₅₀ at an α-1A receptor can be determined by assaying for intracellular calcium. Intracellular calcium can be assayed, without limitation, in HEK293 cells stably expressing an α-1A receptor, such as a bovine α-1A receptor.

For the purposes of the present invention, it is desired to avoid or minimize triggering of α-1 receptors. Even a small critical threshold achieved of undesired α-1 receptor recruitment creates sufficient generalized vasoconstriction, micro-inflammatory change, and/or pro-inflammatory cytokine release to reduce effectiveness of the α-2 receptor induced positive treatment effects. As all α-2 agonists known have a relative affinity for α-2 vs. α-1, this partial affinity is measure by the ratio of α-2 to α-1 receptor induction, where the multiplied product of the degree of selective α-2 affinity—the α-2/α-1 ratio x the concentration C % determines that actual total pool of both α-2 and α-1 receptors induced.

It is a discovery of the present invention that at very low concentrations of highly lipophilic and highly selective α-2 agonists, they still have a sufficiently strong activation of α-2 receptors for IOP efficacy—but with minimal or no cross-activation of α-1 receptors, as α-2 activation becomes very large and α-1 activation so small as to be clinically negligible. The discovered range of necessary high selectivity, high lipophilicity and low concentration completely alters the IOP efficacy and side effect profile of α-2 agonist drugs. Accordingly, when these α-2 agonists are used for the treatment of glaucoma, they greatly reduce IOP and provide eye whitening without significant side effects believed to be associated with α-1 receptors, such as rebound hyperemia.

In some embodiments, compositions and methods of the invention include selective α-2 adrenergic receptor agonists which have Kᵢ for α-2 over α-1 receptors of 1500 fold or greater and have an octanol-water partition coefficient of about Log P 2.50-3.0 adjusted however for topical pH (Log D) to be between 0.75 and 2.20 and preferably 1.0 and 2.20. Tears and intraocular fluids are physiologic at pH 7.4, which is equal to Log P and, according to the precepts of the present invention, confers IOP reduction benefits. Corneal physiology requires a delicate and different octanol-water Log value (called Log D, determined by the pH of the formulation), so that the formulations are able to not only penetrate the lipophilic corneal epithelium and inner endothelium, but also penetrate the hydrophilic middle stromal layer.

In yet other embodiments, compositions and methods of the invention include selective α-2 adrenergic receptor agonists which have Kᵢ for α-2 over α-1 receptors of 1000 fold or greater and are at a concentration from between about 0.0035% to about 0.035% weight by volume.

It is further preferred that α-2 agonists preferably target α-2a receptors as compared to α-2b or α-2c receptors.

Brimonidine, guanfacine, guanabenz, dexmedetomidine and fadolmidine are some of the sufficiently highly selective α-2 agonists to satisfy the selectivity requirement. However, of these highly selective α-2 agonists, only dexmedetomidine satisfies other additional preferred formulation characteristics of the present invention, such as lipophilicity. Accordingly, novel provided formulations of dexmedetomidine are believed to constitute an effective α-2 next generation super drug for the treatment of glaucoma. It is believed that new α-2 agonists can be synthesized to meet the requirements of the present invention.

### Lipophilicity

Lipophilicity may be measured, for example, using known measurements, such as log P (log K_{OW}) derivation of the octanol-water partition coefficient and/or, a closely related coefficient, XLogP3-AA. See, for example, Tiejun Cheng et al, Computation of Octanol-Water Partition Coefficients by Guiding an Additive Model with Knowledge, J. Chem. Inf. Model., 2007, 47 (6), pp 2140-2148. These measurements represent the intraocular lipophilicity value of topical drugs for intraocular delivery (i.e., once the drug permeates into the anterior chamber and is at a pH of 7.4). A person of ordinary skill in the art is well familiar with these measurements.

It is believed that lipophilicity of an α-2 agonist compound is related to pH: for weak base α-2 agonists, such as brimonidine and dexmedetomidine, the more alkaline pH, the more the equilibrium between ionized base releasing H+ and nonionized base shifts to the left (nonionized), resulting in a more lipophilic compound. This is particularly true for α-2 agonists with pKa values of near or greater than 7.0, as is the case for brimonidine and dexmedetomidine. This is because at a more alkaline pH, more of the compound is present in a non-ionized form, and conversely therefore, at more acidic pH more of a drug is ionized and less lipophilic. Usually, Log P and/or XLogP3-AA are measured when the formulation at issue is or will be at the physiologic pH of about 7.4. However, brimonidine becomes hydrophilic (negative Log P value) below pH 6.7, and corneal penetration requires drugs with a highly specific degree of lipophilicity (not too little and not too great) which depends on each drug's pKa, Log P value, and classification as weak acid or base. It was discovered in prior art that increasing the pH results in a better lipophilicity profile, making brimonidine mildly lipophilic on topical instillation and resulting in a better corneal penetration. This brimonidine formulation is commercially known as Alphagan P®, pH specified to between 7.4 and 7.8. However, dexmedetomidine is highly lipophilic with a highly selectable range from just above the brimonidine's lipophilicity at pH 7.4 to 7.8 (Log D=0.50) starting at Log D of 0.75 at pH 4.0 and increasing to Log D 2.96 at pH 7.4, defining the entire useful known range of preferred lipophilicity within which virtually all ophthalmic drugs will have a preferred Log value.

The preferred Log P (and XLogP3-AA) values—those that define intraocular performance according to the present invention—that are suitable for the purposes of the invention are between about 2.00 and 5.00; and more preferably, between about 2.75 and 3.50. The Log P value helps define the intrinsic intraocular lipophilicity where pH is about 7.4. In this range, an α-2 agonist is highly lipophilic and may more easily penetrate lipophilic cell membranes where α-2 receptors are found. Further, while not wishing to be held to a particular theory, it is possible its binding affinity to such receptors is increased and, because of depot absorption by and diffusion from lipophilic intraocular structures (such as ciliary processes where α-2 agonists confer their primary effect, iris pigment and retinal pigment epithelium), its concentration and efficacy are enhanced.

If the selectivity of a specific α-2 agonist is substantially above 1600:1 (for example, 2000:1), then it is possible that this agonist may be effective for the purposes of the invention even if it is less lipophilic (has a slightly lower octanol-water partition coefficient), and vice versa.

In some embodiments, dexmedetomidine, or another synthesizable selective α-2 adrenergic receptor agonist, has Log P at pH 7.4 of about 3.10; preferably, between about 2.0 and 5.00; and more preferably between about 2.75 and 3.50 for intraocular lipophilicity. It was unexpectedly found that a relatively acidic pH provides a much stronger clinical effect than a higher pH, which is directly contrary to the commonly accepted understanding of brimonidine which led to formulating Alphagan P®. For example, a 0.022% solution of dexmedetomidine provides about a peak 15% reduction in an intraocular pressure (IOP) at pH 7.4, and provides as high as 38% reduction in IOP at pH 5.0.

As Log D refers to a lipophilicity value at a given pH, this measurement is especially useful to determine the level of topical lipophilicity and resultant corneal permeability of a topical composition according to the principles of the present invention. Corneal permeability is a complex event, which may be affected by polar surface area, H⁺ donor activity, bond rotation, and active transport phenomenon. It is a discovery of the present invention that the Log D values of between about 0.75 and about 2.20, and more particularly between about 1.00 and about 1.50, corresponding to pH ranges of 4.0 to 6.2 and 4.7 to 5.3. respectively, are preferred for increased corneal permeation of dexmedetomidine and by prophetic example for other similar α-2 agonists. Adding to the complexity, the cornea is a lipophilic-hydrophilic-lipophilic sandwich, where, as a discovery of the present invention, even nonbuffered topical pH can alter the entire corneal pH for as long as 15 minutes before physiologic equilibration to tear, tissue and intraocular fluid pH of 7.4, and even more so when the pH is buffered. In a preferred embodiment for dexmedetomidine, a formulated pH of 5.0 with or without a buffer is delivered topically, rendering the entire cornea relatively more acidic for a period of several minutes, and thereby increasing the stromal diffusion at this more hydrophilic pH range.

For any given drug, an optimal lipophilicity exists to maximize requisite penetration into the lipophilic cornea surface epithelium and, to a lesser extent, inner layer endothelium. If a drug is too hydrophilic, the epithelium becomes an impenetrable barrier. If a drug is too lipophilic, the drug cannot pass through the more hydrophilic stroma. For a majority of drugs a general trend of Log P values from 2.0 to 3.0 is thought to be the best range of lipophilicity, though some of the best absorbing drugs range from 1.00 to about 2.50 (Li et al, A Study of the Relationship between Cornea Permeability and Eye irritation using Membrane-interaction QSAR Analysis; Toxicological Sciences 88(2), Fig. 4-5, 434-446). Since each drug is unique in that it has its own Log P, and is not always amenable to stable Log D/pH manipulation, little is known about how each drug might be further optimized for topical delivery. For brimonidine, which is essentially only a very mildly lipophilic drug at neutral or alkaline pH that in fact becomes hydrophilic at a flexion point at or below a pH of 6.7 or less, increasing pH above this flexion point results in lipophilic Log D values, such that at pH 7.4, brimonidine's Log D is 0.49-0.79 (ACD Labs, Drug Bank respectively).

For dexmedetomidine, published studies show modest IOP lowering efficacy using a pH of 6.4 in normotensive rabbit eyes in a commonly used rabbit model with phosphate buffered delivery. Only after induced high levels of ocular hypertension could significant IOP reduction be obtained, while it is generally recognized all glaucoma drugs have an IOP reducing efficacy that increases with starting baseline IOP. A surprising discovery of the present invention is that instead of an expected improvement in effectiveness at pH 7.4 for dexmedetomidine, there was nearly complete loss of IOP lowering effect. Further, it was additionally discovered that a dramatic increase in effectiveness occurred by lowering the pH to 5.0 for dexmedetomidine: the IOP lowering effects in a normotensive eye went from about 15% (at pH of 7.4) to 38% (at pH of 5.0)

Wishing not to be held to any specific theory or mechanism, this completely surprising and unexpected finding suggests that the initial attempt to utilize a more alkaline pH was not transiently reversed within seconds of administration after epithelial contact, and instead was maintained for at least minutes or tens of minutes. Further, while wishing not to be held to a particular theory, the alkaline pH most likely and unexpectedly resulted in too high a degree of lipophilicity to diffuse out of the epithelium through the stroma. The surface drug may have become trapped in the epithelium, where absorption would be expected to increase, and once saturated, was no longer able to absorb because it was too lipophilic to penetrate the hydrophilic stroma.

As a result of this finding, a discovery of the present invention is that highly lipophilic and highly selective α-2 agonists that are weak bases are too lipophilic for optimal delivery at a neutral or alkaline pH; and if a drug's profile results in Log D values of 0.50-1.50 at lower pH ranges acceptable for ophthalmic delivery (above 4.0 and preferably at or above 4.50), an optimized formulation pH for that drug can be obtained.

As the anterior and posterior portions of the eye contain important highly lipophilic structures, greater α-2 agonist membrane penetration and absorption become possible. As pigment is highly lipophilic, structures that may facilitate IOP reduction via improved drug absorption from the aqueous include:
1) pigmented ciliary body, and particularly ciliary process where aqueous is formed and where the outer layer is highly pigmented;
2) iris pigment epithelium, where drug diffusing past the iris level is much more highly absorbed via highly lipophilic α-2 agonists such as dexmedetomidine at Log P 2.96 than brimonidine at its Log P of 0.49, and where such values represent a logarithmic differential, after which a general drug diffusion increases exposure to the ciliary processes;
3) trabecular meshwork, a main outflow channel where α-2 receptors are known to exist; and
4) retinal pigment epithelium, where similar to the other pigmented structures increased drug absorption and later general diffusion may increase the concentration available at the posterior retinal surface where the optic nerve and nerve fiber layers may achieve more effective levels of known neuroprotective effects of dexmedetomidine over less lipophilic but also known neuroprotective brimionidine.

For a variety of reasons described above, such improved topical delivery results in equal or, in many formulations, a greater IOP lowering effect vs. time than that of such α-2 agonists as clonidine (0.3%), apraclonidine (0.5%-2%) or brimonidine (Alphagan P® (0.10%-0.20%)). Further, it is expected that provided dexmedetomidine formulations will result in a lesser rebound redness effect than brimonidine formulations due to the lower concentration and reduced α-1 receptors induction with the provided dexmedetomidine formulations. Both peak and duration of the effect appear to be improved vs. brimonidine. See, formulations listed in Table 3 in Example 1 below.

Specifically, the IOP was reduced with a three hour value of up to 42.4% using formulations 3 and 4, and over 50% using formulation 7; and a six hour value of 35.5% using formulation 4, and over 50% using formulation 7 ( ). The peak for dexmedetomidine appears to be about 3-3.5 hours vs. 2 hours for Brimonidine. Published results for brimonidine 0.2% used bid on eyes with a mean IOP of 17 (low tension glaucoma) showed a mean IOP reduction of 18.1% vs. over 25% for the present invention after 1 day of use (healthy volunteer, mean IOP 17, brimonidine results include long term use) in one study and 6.6% in another (Krupin, J. M. et al, A randomized trial of brimonidine versus timolol in preserving visual function: Results from the Low-pressure Glaucoma Treatment Study, American Journal of Ophthalmology 2011; 151: 671-681).

Alpha-2 agonists have a dual mechanism of IOP lowering: they both reduce aqueous humor production and stimulate aqueous humor outflow through the uveoscleral pathway (Toris C B, Camras C B, Yablonski M E, Acute versus chronic effects of brimonidine on aqueous humor dynamics in ocular hypertensive patients, Am J Ophthalmol. 1999; 128:8-14). For example, the predominant effect of short-term brimonidine treatment is inhibition of aqueous production, whereas the predominant effect of chronic treatment is stimulation of aqueous humor outflow through the uveoscleral pathway. Id.

The novel α-2 agonist glaucoma drug formulations allow a greater efficacy at much lower topical concentrations with significantly reduced side effects, such as allergic reaction and rebound hyperemia (redness). In fact, an additional whitening occurs that further improves cosmesis and patient compliance for the preferred embodiment of dexmedetomidine at a concentration range of 0.007%-0.075%, vs. when the formulations of 1-2% for apraclonidine, 0.3% for clonidine, or 0.10-0.20% brimonidine are used.

An expert in the art may readily formulate selective α-2 agonists to have a Log P value (i.e., a measure of intraocular efficacy) at or significantly above 2.0 to 3.0, and to have a significantly lower Log D value (a measure of corneal permeation) at a lower pH by synthesizing a weak base, most easily as a derivative of dexmedetomidine. It will be therefore well understood by any expert in the art that the present invention provides a means to formulate improved α-2 agonist glaucoma drugs via acidified (pH below 6.4 and preferably below 5.4) topical formulation for the drugs which have high Log P values of about 2.0-3.5; are basic drugs such as an imidazoline; have pKa values of about 6.5 or greater; and have α-2/α-1 high selectivity of 1000:1 or preferably 1500:1 or greater, but the exact values may vary slightly for each individual synthesized drug.

Table 1 provides known XLogP3-AA values (a more accurate Log P) and α2/α1 binding affinities for several α-2 agonists.

TABLE 1

| α-2 Agonist | XLogP3AA | α2:α1 |
|---|---|---|
| Brimonidine (0.15% pH 6.6-7.4; 0.10% pH | 0.6-1.8 | 976 |
| 7.4-8.0) | | |
| Guanfacine | 2.0 | |
| Guanabenz | 1.7 | |
| Dexmedetomidine | 3.1 | 1620 |
| Fadolmidine pivalyl prodrug ester | 1.8 | |
| Fadolmidine | 1.2 | |
| Methoxamine | 0.5 | |
| Oxymetazoline | 2.9 | 50 |
| Epinephrine | −1.4 | |
| Clonidine | 1.6 | 200 |
| Apraclondine | 1.3 | 150 |
| Mivazerol | 1.1 | |
| Xylazine | 2.8 | 160 |
| Methyl Dopa | −1.9 | |
| Lofexidine | 2.6 | <300 |

Table 1 demonstrates that among the listed α-2 agonists, only dexmedetomidine has an acceptable combination of high lipophilic XLogP3-AA and highly selective α2:α1 coefficient. However, it is possible that formulations including other α-2 agonists can be achieved which meet the defined requirements of the present invention in both selectivity and lipophilicity categories.

### Acidity

It is preferred that the compositions of the present invention be at an acidified pH of between about 4.0 and about 6.2; preferably between about 4.5 and about 6.0; and even more preferably between about 5.0 and about 5.3. In one embodiment, the decrease in pH from 7.4 to 5.0 was discovered to potentiate glaucoma hypotensive effects of dexmedetomidine formulations from about 15% to about 38% in a normotensive eye—a very high level of reduction rarely found even with prostaglandin class glaucoma drugs and much greater than found for brimonidine or its alkaline formulation as Alphagan® P.

Unless explicitly stated otherwise, when the present application refers to a pH of a formulation of the invention, it refers to the final pH of the formulation. It is to be distinguished from the solution used as diluent, which may have a higher pH than the final pH of the formulation solubilized within the diluent.

Normally, α-2 agonists are formulated as salts selected to improve solubility at an acidic pH, and typically achieve an acidic pH when dissolved in water (pH of about 3.5-6.0). It is a surprising discovery of the present invention that a pH of 6.5 or greater is preferred for topical glaucoma therapy because of the exponential decrease in solubility in this range to about 0.022% or less, below known effective concentrations of α-2 agonist glaucoma drugs.

However, the acidified pH is also less than the pKa of dexmedetomidine at 7.1, thereby increasing the percentage of drug in ionized, more hydrophilic and less lipophilic form, as reflected by the Log D value decrease from 2.82 at pH 7.0 to as low as 0.73 at pH 4.0. The acidified pH, particularly in the 5.0 to 5.3 range, increases and potentiates corneal penetration of the provided compositions. Once intraocular, equilibration to physiologic intraocular pH of 7.4 alters the equilibrium back to the non-ionized lipophilic Log P 3.0, where the heightened lipophilicity and α-2 selectivity of dexmedetomidine (a preferred α-2 agonist) at intraocular pH levels of about 7.4 (Log P value) increases intraocular peak hypotensive effect as well as duration of the effect versus other α-2 agonists.

Generally, one can vary the level of acidification of the compositions of the invention to achieve a preferred degree of topical lipophilicity and balance desired corneal permeation ideal range for the present invention. For dexmedetomidine, the preferred Log D value is between 0.75 and about 2.2.

For example, at a final pH of between 6.4 and about 7.4, a sufficiently high octanol-water partition coefficient exists that prevents optimal/adequate corneal stromal permeation of dexmedetomidine. This is different from the less lipophilic brimonidine which is hydrophilic at lower pH. However, at a pH of between 4.0 and 6.2, and more preferably 5.0 and 5.5, selective α-2 agonists, particularly those with neutral pKa which are basic drugs, e.g., dexmedetomidine, have a reduced Log D relative to their physiologic pH value and achieve desired topical lipophiliicity, both of which are preferred for the purposes of the present invention.

At a diluent pH of 7.1, the lipophilicity of dexmedetomidine is significantly increased to a Log P of nearly 3 versus 2.20 at pH 6.2, and 0.79 at pH 4.0. Therefore, a large range of potential formulation Log D values exists for determining the optimal formulation range for the present invention. Dexmedetomidine, a preferred α-2 agonist, at a pH of between about 4.0 to about 6.2, and more preferably from about 5.0 to about 5.3, has been experimentally determined to provide greatest efficacy.

### Corneal Permeation

The compositions of the present invention have an improved α-2 intraocular efficacy and corneal permeation. As a rough rule of thumb, if a drug's efficacy, once the drug is inside the eye, increases 50% but the drug's permeation decreases 50%, the net effect remains unchanged. Without wishing to be bound to a specific theory, it is believed that the improved efficacy and corneal permeation of the compositions of the present invention is largely due to their optimized intraocular lipophilicity and optimized topical lipophilicity.

Certain additives, including weak acids, chelating agents, and cyclodextrins, can increase the corneal permeation. In particular, it has been found that mucoadhesive additives can improve active drug performance for a preferred embodiment of dexmedetomidine in a number of ways:
1) reduce loss to nasolacrimal duct clearance, thereby increasing the amount of remaining drug per unit time;
2) increase the direct contact time of the active drug with the cornea;
3) increasing the time when the drug is shielded from neutralization by tears (which are at a physiologic pH), thereby allowing for an improved corneal permeation; and
4) safely allow for an increased concentration of the drug, due to decreased clearance via the nasal lacrimal duct and then into systemic circulation.

Once the additive components are disassociated from the active agent intraocularly (e.g., dexmedetomidine), the active agent of the present invention has intraocular efficacy associated with two variables: 1) proportional to the degree of α2:α1 selectivity and, according to the principles of the present invention, and 2) proportional to the degree of intraocular lipophilicity (Log P).

The preferred combination of α2:α1 selectivity of 1600:1 and Log P of about 2.89 to about 3.1 is believed to increase the α-2 agonist's membrane permeation to and within α-2 receptors in ciliary processes, as well as the highly lipophilic iris pigment epithelium adjacent to the ciliary processes, possibly the trabecular meshwork, allowing to reach endothelial cells lining Schlemm's canal and/or other α-2A receptors identified within such meshwork; retinal surface concentration via retinal pigment epithelium absorption and diffusion along the inner plexiform neuronal layer, where α-2 agonists are known to suppress excitotoxic glutamate elevation found in glaucoma and other neuronal degenerative conditions and where α-2 receptors are known to populate these layers. The provided formulations of the present invention generally facilitate the α-2 agonist's target interaction in the highly lipophilic iris and retinal pigment epithelium with subsequent diffusion, and may increase its binding affinity with the α-2 receptor surface, resulting in a longer duration of therapeutic effects.

### Solubility

The solubility of α-2 agonists decreases exponentially at an increased pH. Table 2 illustrates the relationship between pH and solubility in water for dexmedetomidine. It shows that the soluble concentration of dexmedetomidine falls exponentially with higher pH. For pH of 4.0-6.0 a very high degree of solubility exists.

TABLE 2

| | solubility | max soluble | |
|---|---|---|---|
| pH solution* | (mg/ml) | concentration | BSS |
| 6.0 | 1.953 | 0.195% | |
| 6.4 | ~0.60 | 0.060% | |
| 7.0 | 0.224 | 0.023% | ≧0.10% |
| 7.4 | ~0.150 | 0.015% | |
| 8.0 | 0.134 | 0.013% | |

BSS = Balanced Salt Solution

In some embodiments of the present invention, it may be necessary to improve (i.e., increase) the solubility of α-2 agonists. A greater solubility has a number of advantages, including but not limited to an ability to achieve higher concentrations, enhanced stability at storage at cold temperatures, etc. Because the desired concentration of suitable α-2 agonists is very low, and discoveries of the present invention allow formulations with much greater solubility, the desired concentrations are easily achieved particularly in the preferred acidic pH range where such solubity increases exponentially with decreasing pH for weak base α-2 agonists such as the preferred embodiment of dexmedetomidine.

It is a surprising discovery of the present invention that α-2 agonists, and more specifically, dexmedetomidine, are rendered more soluble by constituents of a balanced salt solution. The terms “salt” and “constituent of a balanced salt solution” are used interchangeably for the purposes of the present invention. They are a subset of agents that improve solubility of the inventive formulations. It was discovered the addition of a balanced salt solution, and more particularly of sodium citrate dihydrate at 0.17% (as part of Alcon® balanced salt solution) contributed to over 500% increase in the solubility at pH 7.1, allowing the maximum concentration to increase from 0.022% to greater than or equal to 0.10%. Though in the acidified pH range of the present invention solubility is greatly enhanced, it may be desirable in some formulations for some pH values to further solubilize the drug and obtain added stability. Thus, in one embodiment of the present invention, dexmedetomidine is rendered soluble up to or beyond 0.1% by adding constituents of a balanced salt solution. In a preferred embodiment, these constituents include any combination of one or more of the following: sodium citrate dehydrate, sodium acetate, and calcium salt. In a more preferred embodiment, the concentration of sodium dehydrate is about 0.17%; the concentration of sodium acetate is about 0.39%; and the concentration of calcium salt is about 0.048%.

The most preferred agent that improves solubility is a citrate salt. Citrate salt acts as a preservative and a corneal penetration enhancer.

Other agents that improve solubility which may be used for the purposes of the present invention include, but are not limited to, methanesulfonate (mesylate), hydrobromide/bromide, acetate, fumarate, sulfate/bisulfate, succinate, citrate, phosphate, maleate, nitrate, tartrate, benzoate, carbonate, pamoate, borate, glycolate, pivylate, sodium citrate monohydrate, sodium citrate trihydrate, sodium carbonate, sodium EDTA, phosphoric acid, pentasodium pentetate, tetrasodium etidronate, tetrasodium pyrophosphate, diammonium ethylenediamine triacetate, hydroxyethyl-ethylenediamine triacetic acid, diethylenetriamine pentaacetic acid, nitriloacetic acid, and various other alkaline buffering salts, polyanionic (multiple negatively charged) compounds, such as methylcellulose and derivatives, particularly carboxymethyl cellulose (CMC); and/or addition of cyclodextrins and/or their derivatives, particularly (2-Hydroxypropyl)—beta-cyclodextrin; certain solvents such as Tween 20, Tween 80, polyvinyl alcohol, propylene glycol and analogues or derivatives thereof; certain osmotic agents, such as mannitol or sucrose, HPMC or analogues and/or derivatives thereof, or certain chelating agents.

It is well within a skill of a skilled in the art to determine the amounts and concentrations of the agents improving solubility, and thereby consider such agents for testing if and when it is desired to further improve formulation stability, such as during long term high temperature stability analysis.

### Compositions and Methods of the Present Invention

Compositions and methods of the inventions encompass all isomeric forms of the described α-2 adrenergic receptor agonists, their racemic mixtures, enol forms, solvated and unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977) which is incorporated herein by reference).

As long as a particular isomer, salt, analog, prodrug or other derivative of a suitable selective α-2 adrenergic receptor agonist functions as a suitable selective α-2 agonist, it may be used for the purposes of the present invention.

When choosing a particular α-2 adrenergic receptor agonist, one may take into account various considerations including any possible side effects and other systemic reactions.

The compositions of the present invention are preferably formulated for a mammal, and more preferably, for a human. In one embodiment of the invention, the compositions are delivered as ophthalmic solutions into the eyes. The invention also contemplates topical compositions which include, but are not limited to, gels and creams. They may also include additional non-therapeutic components, which include, but are not limited to, preservatives, delivery vehicles, tonicity adjustors, buffers, pH adjustors, antioxidants, tenacity adjusting agents, mucoadhesive agents, viscosity adjusting agents, and water.

The compositions of the invention may include various inactive ingredients commonly used in formulating topical compositions and that may improve stability of the formulation. For example, the compositions of the invention may include alcohols and/or surface active agents, including but not limited to polyglycol ether, polyethylene glycol-nonphenol ether, polyethylene glycol sorbitan monolaurate, polyethylene glycol sorbitan monooleate, polyethylene glycol sorbitanmonooleate, polyethylene glycol sterarate, polyethylene glycol polypropylene glycol ether, polyvinyl alcohol, polyvinyl pyrrolidine, PEG and its derivatives, including but not limited to PEG 4000 or PEG 6000, in a total amount of 0.05% to 5% by mass of the composition.

In some embodiments, the compositions of the invention may include acids or monoglycerides of fatty acids having 8 to 12 carbon atoms, which when in 0.5-1.5 M, and preferably equimolar concentration to the alpha 2 agonist may improve corneal permeation via ion pair formation; or antioxidants such as ion-exchange/photooxidation stabilizing agents, including but not limited to citric acid, sorbic acid, boric acid, caprylic acid, glyceryl monocaprylate, glyceryl monocaproate, glycerol monolaurate, sodium metabisulfite.

In some embodiments, the compositions and methods of the present invention may include chelating agents that further improve stability, including but not limited to ethylenediaminetetraacetic acid (EDTA) and structurally related acids and even more preferably citric acid or its salt. In some embodiments, the chelating agents are present at a concentration of between 0.02% and 0.2% weight/vol.

Preservatives include, but are not limited to, benzalkonium chloride (BAK), methylparaben, polypropylparaben, chlorobutanol, thimerosal, phenylmercuric acetate, perborate, or phenylmercuric nitrate.

Delivery vehicles include, but are not limited to, polyvinyl alcohol, polyethyleneglycol (PEG) and its analogues, povidone, hydroxypropyl methyl cellulose, poloxamers, carboxymethyl cellulose (CMC), hydroxyethyl cellulose and purified water. It is also possible to use a physiological saline solution as a major vehicle.

Tonicity adjustors include, but are not limited to, a salt such as sodium chloride, potassium chloride, dextran, cyclodextrins, mannitol, dextrose, glycerin, or another pharmaceutically or ophthalmically acceptable tonicity adjustor. In some embodiments, the tonicity modifying agents are present at a concentration of between 0.5% and 5% weight by volume.

The compositions of the present invention may comprise corneal permeation enhancing agents which include, but are not limited to, preservatives, cyclodextrins, viscosity enhancing agents, and ion-channel enhancing agents. In some embodiments of the invention, a corneal permeation enhancing agent may be selected from the group consisting of BAK at 0.01% to 0.02% weight by volume, EDTA at 0.01% weight by volume, caprylic acid, citric acid, boric acid, sorbic acid and/or salts, derivatives, and analogues thereof, where citric acid or its salt is a preferred embodiment.

Many of the listed additives (for example, BAK, EDTA, etc) may serve more than one purpose: for example, they can serve as both preservatives and corneal permeation enhancing agents (e.g. BAK), or solubilizing, preservative, and corneal permeation enhancing agents (e.g. citrate).

In some embodiments, the compositions and methods of the present invention may include viscosity agents and/or agents increasing solubility and/or stability, including but not limited to polyvinylpyrrolidone, methylcellulose, hydroxypropylcellulose, CMC, CMC sodium salt, gelatin, cellulose glycolate, sorbitol, alpha-cyclodextrin and/or other cyclodextrin derivatives, niacinamide, etc. In some embodiments, these agents are present at a total amount of 0.05% to 5% by w/v.

In a preferred embodiment, the amount of CMC is between about 0.05% and about 5%, and more preferably, between about 0.1% and about 0.3% weight by volume.

In a preferred embodiment, the amount of cyclodextrin is between about 0.1% and about 20%, and more preferably, between about 0.2% and about 0.5% weight by volume. In another preferred embodiment, the preservatives concentrations are: BAK 0.02%, or BAK 0.01% and EDTA 0.01%, where all units are weight by volume. In another preferred embodiment, the caprylic acid concentration is equimolar to that of dexmedetomidine, adjusted to optimize pH at about 4.0-6.2.

The compositions of the invention may also comprise a solubility stabilizer which preferably contains an anionic component, such as CMC, HPMC, or peroxide class preservatives. The solubility stabilizer allows one to achieve greater penetration of lipophilic membranes. In a preferred embodiment, the solubility stabilizer comprises a stabilized oxychioro complex, chlorite, and sodium perborate as preservative, or BAK as preservative.

Buffers and pH adjustors include, but are not limited to, acetate buffers, carbonate buffers, phosphate buffers and borate buffers. It is understood that various acids or bases can be used to adjust the pH of the composition as needed. pH adjusting agents include, but are not limited to, sodium hydroxide and hydrochloric acid. Because of the Log D reduction via acidified pH of the α-2 agonists of the present invention, and the maintenance of such pH reduction for minutes or tens of minutes contributing to greater corneal permeation, it is highly desirable to provide a buffer with the 4.0-6.2 pH range. Acetate and citrate buffers are believed to be especially effective.

Antioxidants include, but are not limited to, sodium metabisulfite, sodium thiosulfate, acetylcysteine, butylated hydroxyanisole and butylated hydroxytoluene.

To make the topical compositions of the present invention, one can simply dilute more concentrated solutions of selective α-2 agonists, using methods known in the art with diluent of normal saline or a balanced salt solution consisting of one or more of the above electrolytes or tonicity enhancing agents and preferably one or more of the above weak acids and or their salts to achieve a formulated pH of 4.0 to 6.2, and more preferably 4.7-5.3. The precise method of carrying out the dilutions is not critical. Any commonly used diluents, including preservatives described above in the application, suitable for topical solutions can be used.

The preferred compositions of the invention may include the following components:
dexmedetomidine at 0.007%-0.070%, most preferably 0.022% to 0.035% weight by volume;
Poloxamer, particularly Poloxamer 407 (Pluronic® F127) at 1%-10%, most preferably 2%-4%;
PEG, particularly PEG 6000 or 4000 at 0.5%-5%; most preferably 1%-3%;
mannitol at 1%-5%; most preferably 4%;
CMC at 0.1%-0.5%, most preferably 0.15%;
propylene glycol or polyvinyl alcohol at 1%-5%, most preferably 1.5%;
sodium acetate buffer to pH 4.0-6.2; preferably at 5.0; at 1-100 mM, 5-10 mM preferred)
caprylic acid, preferably equimolar to dexmedetomidine; and
BAK at 0.01%-0.02%; preferably at 0.01%;
adjust with NaCl to 280-320 milliosmoles.

In some embodiments, the invention provides the following compositions:
1. dexmedetomidine 0.0025%-0.035%;
   diluent: balanced salt solution or 0.9% saline solution;
   0.01% BAK;
   acetate or citrate buffer 1 mM-100 mM; 5-10 mM preferred; and
   final pH of about 4.5-5.4, more preferably 5.0.
2. dexmedetomidine 0.035%;
   diluent: balanced salt solution or 0.9% saline solution;
   0.02% BAK;
   Poloxamer 407 2%; and
   final pH 5.0.
3. dexmedetomidine 0.0025%;
   diluent: balanced salt solution or 0.9% saline solution;
   CMC (carboxymethylcellulose) 0.15%;
   0.01% BAK;
   0.01% EDTA; and
   final pH 4.7.
4. dexmedetomidine 0.070%;
   propylene glycol 1.0%;
   PEG 2%;
   Poloxamer 407 4%;
   0.02% BAK;
   diluent: saline 0.70% (7 mg/ml) NaCl (optionally) and any or all of:
   KCl 0.05% (0.50 mg/cc), CaCl₂ 0.07% (0.075 mg/cc), MgCl₂ 0.0037% (0.037 mg/cc); and
   final pH 5.0.
5. dexmedetomidine 0.04%;
   propylene glycol or polyvinyl alcohol, 1.5%;
   mannitol as osmotic agent (as needed to create 290 mOsm) about 1%;
   acetate or citrate buffer;
   CMC 0.15%;
   0.02% BAK;
   diluent: saline 7 mg/cc (0.70%-0.90%) NaCl optionally and any or all of:
   KCl 0.05% (0.50 mg/cc), MgCl₂ 0.0037% (0.037 mg/cc); and
   final pH 4.75.
6. dexmedetomidine 0.022%;
   diluent: balanced salt solution or 0.9% saline solution;
   0.01% BAK;
   Sodium citrate dehydrate 0.2%;
   Poloxamer 407 6-8%;
   Propylene glycol 1.5%;
   PEG 6000 2.5% (increase gelation temp) or
   PEG 4000 2.5% (decrease gelation temp)
   Acetate buffer 1-100 mM, preferably about 5-10 mM; and
   final pH to 4.7-5.7.

In the most preferred embodiment, the compositions of the invention include the following ingredients and are at pH of 5.0:
a. dexmedetomidine at a concentration of 0.035%;
b. pvpK29-32 2%;
c. benzyl alcohol 0.50%;
d. polysorbate 80 0.75%;
e. propylene glycol 0.50%;
f. potassium sorbate 0.150%;
g. Poloxamer 407 2%-8%;
h. citric acid 0.15% or as needed to achieve pH; and
i. NaOH, HCl for final adjustment of pH to 5.0

In the most preferred embodiment, the compositions of the invention include
a. dexmedetomidine at a concentration from between about 0.005% to about 0.05% weight by volume, more preferably 0.022% to 0.035%;
b. BAK 0.02% or BAK 0.01%; and
c. Poloxamer 407 2%,
wherein said pharmaceutical composition has a final pH of about 4.7-5.3.

The following Examples are provided solely for illustrative purposes and are not meant to limit the invention in any way.

### Example 1

### Effect of Inventive Formulations on Intraocular Pressure Experimental Design

A variety of formulations and variations as described above were tested for intraocular pressure reducing efficacy. The experimental design included two drops of drug instilled into one or both eyes, and intraocular pressure testing using slit lamp goldman applanation tonometry, where fluorescein was first instilled. Two initial readings were taken and discarded to ensure no blepharospasm artifact and proper thin but complete fluorescein applanation rings by dabbing away any excess fluorescein noted. The subsequent readings were repeated three to five times, with all readings required to be within a deviation of no more than 2 mm from each other. Readings outside of this range were discarded. Baseline was taken from a 24 hour diurnal curve prior to drug administration, comparative time points used for IOP % reduction determination. Readings were taken at various post instillation time points ranging from 2, 3, 4.5, 5, 6, and 23 hours post instillation.

### Experimental Results

Table 3 demonstrates the results of this experiment.

TABLE 3
Inventive Formulations Vs. IOP

| | | Time After | | | | |
|---|---|---|---|---|---|---|
| Formula- | Con. | Administra- | | | Preser- | P |
| tion No. | % | tion (Hours) | pH | Diluent | vative | 407 |
| 1 | 0.035% | 3 | 4.5 | 0.9% NS | 0.01% BAK | — |
| 2 | 0.022% | 3 | 5.0 | 0.9% NS, | 0.01% BAK | — |
| | | | | BSS | | |
| 3 | 0.035% | 3.5 | 5.0 | BSS | 0.02% BAK | — |
| 4 | 0.035% | 3 | 5.0 | 0.9% NS | 0.01% BAK | 2% |
| | 0.035% | 6 | 5.0 | 0.9% NS | 0.01% BAK | 2% |
| 5 | 0.035% | 6 | 5.0 | 0.9% NS | 0.01% BAK | — |
| 6¹ | 0.035% | 3 | 5.0 | 0.9% NS | 0.01% BAK | 4% |
| 7 | 0.035% | 2 | 5.0 | 0.9% NS | 0.01% BAK | 8% |
| | 0.035% | 3 | 6.0 | 0.9% NS | 0.01% BAK | 8% |
| | 0.035% | 6 | 7.0 | 0.9% NS | 0.01% BAK | 8% |
| 8¹ | 0.035% | 3 | 5.7 | 0.9% NS | 0.01% BAK | — |
| 9 | 0.035% | 3 | 7.0 | 0.9% NS | 0.01% BAK | — |
| 10 | 0.022% | 3 | 7.4 | BSS | 0.01% BAK | — |

| | | | | | Decrease in |
|---|---|---|---|---|---|
| | | | | | Redness |
| | | | | | and/or |
| Formula- | | Decrease | Irritation | Sedation | Increase in |
| tion No. | Other | in IOP % | 0-4 | 0-4 | Whiteness |
| 1 | — | 33.0% | 0 | 0.5 | yes |
| 2 | — | 25-35% | 0 | 0 | yes |
| 3 | — | 42.4% | 0 | 0.5 | yes |
| 4 | — | 42.4% | 0 | 0 | yes |
| | — | 35.5% | 0 | 0 | yes |
| 5 | — | 19.7% | 0 | 0.5 | yes |
| 6¹ | — | 25.8% | 1-2 | 0 | yes |
| 7 | PG+ | 54.4% | 0 | 0 | yes |
| | PG+ | 55.9% | 0 | 0 | yes |
| | PG+ | 36.7% | 0 | 0 | yes |
| 8¹ | — | 13.6% | 0 | 0.5 | yes |
| 9 | — | 13.2% | 0 | 0.5 | yes |
| 10 | — | 10.5% | 0 | 0.5 | yes |

¹bilateral treatment
pg = propylene glycol

### Example 2

### Effect of Topical Administration of Dexmedetomidine 0.022% on IOP at pH 5.0, 7.4

### Experimental Design

The purpose of this experiment was to evaluate the effect of topical ocular delivery of dexmedetomidine at pH 5.0 and 7.4 using balanced salt solution as diluent at approximately equal time points about 4.5 hours following administration, 1 week apart.

All IOP measurements for the described experiments were made using Goldmann applanation slit lamp tonometer, with alcaine one drop topically followed by topical fluorescein via strip. Five measurements consecutively were made, with the first two discarded to allow for blepharospasm and fluorescein thickness reduction. Measurements 3-5 were typically within 1 mm, and for the entire range never beyond 2 mm.

### Experimental Results

The results of the experiment are summarized in Table 4.

TABLE 4

| | IOP % |
|---|---|
| pH | (reduction vs. baseline) |
| 7.4 | 10.5% |
| 5.0 | 38% |

### Example 3

### Effect of Topical Administration of Dexmedetomidine at 0.035% at pH 5.0 on IOP Vs. Time with and without Poloxamer 407 2%

### Experimental Design

The purpose of this experiment was to evaluate the time course effect of topical ocular delivery of dexmedetomidine at concentration of 0.035% at pH 5.0 on IOP with and without the addition of Poloxamer 407.

The experiment was designed as follows:

For this experiment, Poloxamer 407 at 2% was combined with the dexmedetomidine 0.035% at pH 5.0 and administered via single dose topical administration of 2 gtts to the right eye, while dexmedetomidine 0.035% at pH 5.0 without Poloxamer 407 was administered to the left eye.

Following the administration, IOP was measured at 3 and 6 hours following dosing. All IOP measurements were made five times consecutively. The first two measurements were discarded to eliminate slight blepharospasm and excess fluorescein that can reduce measurement accuracy. Only the third, fourth, and fifth measurements were used. In all cases, measurements were within 1 mm of each other and had a total range of deviation of less than 2 mm Hg.

### Experimental Results

TABLE 5

| Baseline IOP | 16 mmHg right and left eye | |
|---|---|---|
| Time After | % IOP Reduction | |
| Administration | Right Eye | Left Eye |
| (Hours) | (with Poloxamer 407 2%) | (without Poloxamer) |
| 3 | 42.4 | 37.9 |
| 6 | 35.5 | 19.7 |

As Table 5 demonstrates, both the magnitude and the duration of the effect were increased by adding Poloxamer 407 to the compositions. Further, no stinging or other adverse effects were noted.

### Whitening Effect

In addition to the IOP effect, differential whitening after administration of brimonidine at 0.035% by itself and in combination with poloxamer was also noted in the above experiment as demonstrated in Table 6:

TABLE 6

| Time After Administration | Right Eye | Left Eye |
|---|---|---|
| 5 min | 3.5/4 | 1.5/4 |
| 3 hrs | 2/4 | 0.5/4 |
| 6 hrs | 1/4 | 0/4 |

The whitening scale is from 0 (no effect) to 4 (glistening pearly white eyes).

As Table 6 demonstrates, the addition of poloxamer to brimonidine resulted in a significant whitening of the eye, as compared to administering brimonidine by itself.

### Example 4

### Effect of Topical Administration of Dexmedetomidine at 0.022%, 0.010% and 0.0065% on Cosmetic Appearance of the Eyes

### Experimental Design

The purpose of this experiment was to evaluate effect of dexmedetomidine at concentrations of 0.007%, 0.010% at pH 6.7 (using BSS® 6.7 diluent), and 0.022% at pH 5.0 using normal saline as diluent on cosmetic appearance (i.e., whiteness) of the treated eyes. The experiment was designed as follows:

A drop of dexmedetomidine at 0.0065%, 0.010%, and 0.022% was topically applied to the eyes of an individual. Eye whiteness prior to and after the application was visually measured by the patient on a scale of 0 (white eye, no hyperemia) to 4 (significantly reddened eye, strong hyperemia).

### Experimental Results

TABLE 7

| Conc. | Redness | Eye White Shade |
|---|---|---|
| Baseline | 1.5 | 1 |
| 0.007% | 0.5-1 | 1.5 |
| 0.010% | 0.5 | 2.0 |
| 0.022% | 0.5 | 2.5 |

Conventional Redness Scale 0 (none)-4 (reddest + chemosis),
Eye White Shade 1-4 (whitest): 4 pearly glistening bright white-1 dull, gray/yellow tint

For the 0.01% dexmedetomidine application, pre-application hyperemia was visually estimated by the patient examiner to be 1.25-1.5 out of 4.0. When the treated eyes were examined about 15 minutes after dexmedetomidine at 0.01% was applied, post-application hyperemia was estimated to be <0.5 out of 4.0.

For the 0.0065% dexmedetomidine application, pre-application hyperemia was estimated to be 1.5 out of 4.0 for both eyes. When the treated eyes were examined about 10 minutes after dexmedetomidine at 0.0065% was applied, post-application hyperemia was estimated to be 0-0.5 out of 4.0. The eyes started to whiten about 2 minutes after the application; the maximum whiteness was at about 10 minutes with gradual and slow decline thereafter; and the total duration of the whitening effect was about 4-5 hours.

Dexmedetomidine at 0.022% resulted in whitening effect identical to 0.010%.

The experiment has demonstrated that dexmedetomidine at 0.0065% provides noticeable hyperemia reduction and 0.010% effects slightly greater cosmetic improvement via eye whitening.

### Example 5

### Adding Dexmedetomidine at 0.017% to Xalatan® (Latanoprost) on the Reduction of IOP

### Experimental Design

The purpose of this experiment was to compare the effect on reducing the IOP of a combination of Xalatan® and dexmedetomidine at 0.0133% versus dexmedetomidine at 0.0133% alone.

The experiment was designed as follows:

At 0 hr, a baseline IOP in both eyes of a patient was measured prior to administration, and was about 18 mm Hg. Then, a drop of Xalatan® was applied to the right eye of a patient and a drop of dexmedetomidine at 0.017% was applied to the left eye of the patient. 3 hours after administration, a drop of dexmedetomidine at 0.017% was applied to the right eye of the patient. Measurements of the IOP in both eyes were taken at 0, 3, 5, 8, and 16 hours following the administration.

### Experimental Results

The results of the experiment are summarized in Table 8.

TABLE 8

| Time after | | | % of | % of |
|---|---|---|---|---|
| admin- | IOP left eye | IOP right eye | baseline OPI | baseline IOP |
| istration | (Hg) | (Hg) | (left eye) | (right eye) |
| 0 hr | 18 (initial IOP) | 18 (initial IOP) | 100% | 100% |
| 3 hrs | 11 (dex alone) | 14 (Xalatan ® | 61.1% | 77.8% |
| | | alone) | | |
| 5 hrs | 10 (dex alone) | 8.5 (Xalatan ® | 55.6% | 47.2% |
| | | plus dex) | | |
| 8 hrs | 10 (dex alone) | 7.5 (Xalatan ® | 55.6% | 41.7% |
| | | plus dex) | | |
| 16 hrs | 16 (dex alone) | 16 (Xalatan ® | 88.9% | 88.9% |
| | | plus dex) | | |

The experiment has demonstrated that topically applying dexmedetomidine at 0.017% significantly potentiates the effectiveness of Xalatan® and leads to a prolonged and significant reduction of the IOP. Further, dexmedetomidine at 0.017% alone is highly effective in reducing the IOP.

What is claimed is:

1. A pharmaceutical composition comprising dexmedetomidine at a concentration from between about 0.007% to about 0.07% weight by volume, wherein said pharmaceutical composition has a pH of between about 4.0 and 6.2, and wherein said pharmaceutical composition is effective for the treatment of glaucoma in a patient in need thereof.

2. The pharmaceutical composition of claim 1, wherein dexmedetomidine is at a concentration from between about 0.025% to 0.035% weight by volume.

3. The pharmaceutical composition of claim 2, wherein said pH is between about 5.0 and 5.7.

4. The pharmaceutical composition of claim 1, further comprising a salt selected from the group consisting of citrate, mesylate, hydrobromide/bromide, acetate, fumarate, sulfate/bisulfate, succinate, phosphate, maleate, nitrate, tartrate, benzoate, carbonate, and pamoate.

5. The pharmaceutical composition of claim 4 wherein said salt is citrate at a concentration of from 0.10% to about 0.50% weight by volume.

6. A pharmaceutical composition comprising dexmedetomidine at a concentration from between about 0.02% to about 0.04% weight by volume, wherein said pharmaceutical composition has an octanol-water partition coefficient Log D of between about 0.70 and about 2.20, and wherein said pharmaceutical composition is effective for the treatment of glaucoma in a patient in need thereof.

7. The pharmaceutical composition of claim 6, wherein said octanol-water partition coefficient is between about 1.25 and 2.00.

8. The pharmaceutical composition of claim 6, wherein said pharmaceutical composition further comprises carboxymethyl cellulose, hydroxymethylcellulose, polyvinylpyrridone, or another viscosity enhancer.

9. The pharmaceutical composition of claim 6, wherein said pharmaceutical composition has a pH of between about 4.0 and 6.2.

10. A pharmaceutical composition comprising dexmedetomidine at a concentration from between about 0.02% to about 0.035% weight by volume, further comprising one or more salts selected from the group consisting of citrate, mesylate, hydrobromide/bromide, acetate, fumarate, sulfate/bisulfate, succinate, phosphate, maleate, nitrate, tartrate, benzoate, carbonate, and pamoate, wherein said pharmaceutical composition has a pH of between about 4.0 and 6.2, wherein said pharmaceutical composition has an octanol-water partition coefficient Log D of between about 1.25 and about 2.20, and wherein said pharmaceutical composition is effective for the treatment of glaucoma in a patient in need thereof.

11. The pharmaceutical composition of claim 10 comprising
a. dexmedetomidine at a concentration from between 0.002% and about 0.02% weight by volume;
b. 0.9% normal saline; and
c. a viscosity enhancer, wherein said viscosity enhancer results in the viscosity of the pharmaceutical composition to be about 3.0 cps.

12. The pharmaceutical composition of claim 11 where the pH range is between 4.0 and 6.2.

13. The pharmaceutical composition of claim 10, comprising a buffer at a concentration between 1 mM and 100 mM, wherein said buffer is selected from acetate, citrate, maleate, sorbate, or phosphate buffer.

14. A pharmaceutical composition comprising:
a. dexmedetomidine at a concentration from between about 0.01% and about 0.05% weight by volume;
b. 0.9% normal saline; and
c. a mucoadhesive,
wherein the salinity of said pharmaceutical composition is about 280-320 milliosmoles.

15. The pharmaceutical composition of claim 14 where the concentration of dexmedetomidine is between about 0.030% and about 0.040%.

16. The pharmaceutical composition of claim 14 where said mucoadhesive is selected from carbapols, poloxamers, xanthum gums, and cellulose derivatives, and wherein said mucoadhesive is at a concentration from between about 0.5% and about 10%.

17. The pharmaceutical composition of claim 16 where said poloxamer is Poloxamer 407.

18. A method of treating glaucoma in a patient in need thereof comprising administering to said patient the pharmaceutical composition of claim 1.
